(19) United States
(12) Patent Application Publication
(10) Pub. No.: US 2020/0147216 A1
(43) Pub. Date: May 14, 2020

(54) DISEASE DETECTION AND TREATMENT THROUGH ACTIVATION OF COMPOUNDS USING EXTERNAL ENERGY

(71) Applicants: Thomas J. Lewis, Morristown, TN (US); Jerry Bommer, Franklin, ID (US)

(72) Inventors: Thomas J. Lewis, Morristown, TN (US); Jerry Bommer, Franklin, ID (US)

(21) Appl. No.: 14/836,169

(22) Filed: Aug. 26, 2015

Related U.S. Application Data
(63) Continuation of application No. PCT/US2013/067982, filed on Nov. 1, 2013.
(60) Provisional application No. 61/769,915, filed on Feb. 27, 2013.

(51) Int. Cl. A61K 41/00; A61K 47/64; A61P 35/00

(52) U.S. Cl. CPC A61K 41/0071; A61P 35/00; A61K 47/645; A61K 41/0033

(57) ABSTRACT

Described herein are compounds for the detection, diagnosis, and treatment of specific diseased tissues, including hyper-proliferative tissues such as tumors, and other tissue diseased with microbial and/or infectious species, using energy-activation methods. In particular, compounds sensitive to externally applied energy, including light and/or ultrasound; that also specifically accumulate in diseased target tissue, are provided.

### BACKGROUND OF THE INVENTION

### Field of the Invention

This invention is related to compositions and methods for treatment of disease or ameliorating conditions associated with one or more disease states and/or detection of disease using energy-activated therapy and agents.

### Summary of the Related Art

Energy-activated compounds for the treatment of disease have been known and applied in medicine for several thousand years. However, the scientific basis was likely not well understood until about 1900. Light energy-activated therapy, also known as photodynamic therapy (PDT) has now become an established treatment modality for several medical indications. Notably, in the cases of skin actinic keratosis, several forms of cancer, and blindness due to macular degeneration, PDT has been successful. PDT is the combined application of a compound, known as a photosensitizer or agent that has affinity and specificity for target tissue; and light, at wavelength and intensity that normally does not create any, or at best minimal, cellular response, whereby the interaction of light and agent produces a reactive species, such as a free radical that is cytotoxic.

A photosensitizer is a chemical compound that can be excited by light of a specific wavelength or wavelength range based on the specific absorption of light by the photosensitizer. This excitation is most effective in therapeutic applications, in mammals, when the light is visible or near-infrared light. In photodynamic therapy, either a photosensitizer or a metabolic precursor of a photosensitizer is administered to the subject.

Next, the tissue to be treated is exposed to light suitable for exciting the photosensitizer. Usually the photosensitizer is excited from the ground singlet state to an excited singlet state. It then undergoes intersystem crossing to a longer-lived excited triplet state. Molecular oxygen may be present in tissue with a ground triplet state. When the photosensitizer and an oxygen molecule are in proximity, an energy transfer can take place allowing the photosensitizer to relax to its ground singlet state and create an excited singlet state oxygen molecule. Singlet oxygen will react relatively rapidly with nearby biomolecules. The specific targets depend on the photosensitizer chosen. Ultimately, these destructive reactions will induce apoptosis or necrosis of the tissue cells.

In the early 1970s scientists at Roswell Park Cancer Institute in Buffalo, N.Y. investigated PDT to determine if it might have efficacy against cancer. Activated psoralens killed rogue cells to settle inflammation, but in comparison to the emerging porphyrin class of PDT photosensitizers, they were not potent. This work ushered in intensive study of photosensitizers and elucidated how, when activated, they interact with oxygen to catalyze the production of oxygen free radicals. In addition, early work suggested two medically useful properties of the porphyrins: they accumulate selectively in cancer cells and are activated by red light, which is able to penetrate more deeply into biological tissues than do shorter wavelengths, such as blue light or UVA.

First proof of the anti-cancer action of porphyrins as photodynamic agents occurred at Roswell Park where a mixture of porphyrins was injected into the bloodstream of mice with mammary tumors. The porphyrins were allowed to build up in the tumors as they purged from the healthy tissue over a period of days before shining red light on them. The light activated the porphyrins within the tumor, which transferred their energy to oxygen, creating reactive free radicals. In almost every case, the tumors blackened and died after the light treatment. There were no signs of recurrence.

Photodynamic therapy can be used to treat cancers and, unlike chemotherapy for cancer, the effect of photodynamic therapy can be localized. Specificity of treatment is achieved because light is delivered only to tissues that a physician wishes to treat. In the absence of light, there is no activation of the photosensitizer and no tissue death. Additionally, photosensitizers may be administered in ways that restrict their mobility. For example, the photosensitizer may be applied only to the specific area to be treated. Further, a photosensitizer may be chosen for its ability to selectively bind to or be absorbed by targeted cells.

The major difference between different types of photosensitizers is the parts of the cell that they target. Unlike in radiation therapy where damage is done by targeting cell DNA, most photosensitizers target other cell structures. For example, m-tetrahydroxyphenylchlorin (mTHPC) has been shown to localize in the nuclear envelope and do its damage there. In contrast, aminolevulinic acid has been found to localize in the mitochondria and Methylene Blue in bacterial cell walls.

Photosensitizing agent can be activated either by coherent (laser) or non-coherent (non-laser) light. It is currently accepted that following absorption of light, the photosensitizer is transformed from its ground singlet state (P) into an electronically excited triplet state (3P*, T˜10−2 sec.) via a short-lived excited singlet state (1P*; T˜10−6 sec.). The excited triplet can undergo non-radiative decay, emit a photo with a “red-shift,” or participate in an electron transfer process with biological substrates to form radicals and radical ions, which can produce singlet oxygen and superoxide (O2-) after interaction with molecular oxygen (O2). Singlet oxygen can be produced from molecular oxygen by the transfer of energy directly or indirectly from the activated photosensitizer. Singlet oxygen is one of the agents responsible for cellular and tissue damage in PDT, causing oxidation of the target tissue. There also is evidence that the superoxide ion may be involved. The generation of these cytotoxic agents plays a role in tumor homeostasis and the observed tumor destruction.

A wide array of photosensitizers for photodynamic therapy are now well studied. Some examples include aminolevulinic acid, silicon phthalocyanine Pc 4, (mTHPC), and mono-L-aspartyl chlorin e6 (NPe6). Several photosensitizers are also commercially available, such as Photofrin, Visudyne, and LS11. While photosensitizers can be used for very different treatments, they share certain characteristics: high absorption at long wavelengths; high singlet oxygen quantum yield; low photobleaching; natural fluorescence; high chemical stability; low dark toxicity; preferential uptake in target tissue; and additionally, the photosensitizer should not be harmful to the target tissue until the activating energy is applied.

In PDT, a photosensitizing agent (“photosensitizer”) is delivered to the target tissue and then radiation, most usually light of wavelengths between 250-1000 nm, e.g., 500-800 nm, or 600-700 nm, is applied to the target tissue. Thus, photosensitizing agents are activated by electromagnetic (“EM”) radiation. PDT is efficient in the presence of oxygen. The oxygen radicals, being reactive and short-lived, do not migrate beyond tissue in their immediate vicinity thus, when localized to diseased tissue, do not impact healthy tissue. In contrast to radiation therapy and chemotherapy, PDT has a low mutagenic potential and, except for skin photosensitivity and/or photo-toxicity, show few adverse effects. Therefore, a desirable biological effect of PDT is the destruction of either or both the cells and surrounding vasculature in a target tissue. For example, PDT can be administered as a primary therapy for early stage disease, palliation or treatment of late stage disease, or as a surgical adjuvant for tumors that show loco-regional spread.

An important factor in the successful use of PDT is that light is needed to activate photosensitizers. This important mechanism allows a relatively non-toxic agent to become highly cytotoxic, on demand, presumably when the agent is highly partitioned into target diseased tissue, thus causing minimal damage to healthy tissue. This factor also limits the utility of PDT because most wavelengths of light cannot penetrate through more than one third of an inch (1 cm) of tissue using standard laser technology and low powered LED technology (see, e.g., Lane, Scientific American, 38, (2003). Thus, PDT is limited to application for treatment of tumors on or under the skin, or on the lining of some internal organs. Fiber optic technology has enhanced the access of PDT to more embedded areas of the body, but still presents a real limitation to whole body cancer treatment and for treatment of deeply embedded tumors. Moreover it is less effective in treatment of large tumors and metastasis for the same reason. However, since about 2007 hollow needles have been used by some units to get the light into deeper tissue.

An emerging approach to energy activated therapy is therapy activated by energy other than light. Well designed compounds can be activated by acoustic energy, in particular ultrasonic or sonic energy (see, e.g., Misik and Riesz, Ann. N.Y. Acad. Sci., 899:335-48 (2000); and U.S. Pat. No. 5,817,048), in a process known as sonosensitization. When sonosensitization is applied in a therapeutic mode, it is referred to as Sonodynamic Therapy (“SDT”).

SDT has been used to treat symptoms or improve conditions associated with various disease states, including cancer. Sonodynamic therapy uses synergistic effects of drugs and ultrasound. A sonosensitizing agent may be derived from, or shares structural similarities to, chlorophyll. Such a sonosensitizing agent is usually sensitive to red light and sensitive to ultrasound. Such an agent is thus both a photosensitizing and sonosensitizing agent. The sonosensitizing agent is preferably specifically absorbed in tumor cells and produces cytotoxic moieties upon interaction with “diagnostic” ultrasound. See Primary Clinical Use of Sonodynamic Therapy (SDT) for Advanced Breast Cancer, The “Tumorocidal effect of Sonodynamic Therapy (SDT) on S-180 sarcoma in mice,” June, 2008, Integrative Cancer Therapies.

Ultrasound is a mechanical wave with wavelength ranging from micrometers to centimeters. Consequently, this acoustic field cannot interact directly with the energy levels of molecules, including those associated with the electronic properties of molecules. Therefore, this radiation is perceived as safe, and has very good tissue penetrating ability without major attenuation of its energy. Thus ultrasound has seen many medical uses including diagnostic imaging of soft tissue.

The interaction of ultrasound with bulk liquid may be accompanied by a phenomenon of cavitation that leads to enormous concentration and conversion of sound energy. In so-called inertial cavities, gas bubbles that grow to the size of the wavelength of the sound energy may expand before violently collapsing, on a microscopic level. The temperature and pressure within the imploding cavities can reach such extreme levels that chemical reactions are induced within the surrounding bubble that include the generation of photons, an emission known as sonoluminescence. In addition to photons, free radicals are known to form in the cavitation bubbles that are able to react with solutes, for example, sonosensitizers, to produce products similar or the same as those formed by the interaction with light.

The variability of photodynamic sensitizer's response to ultrasound may be explained by differences in molar absorption coefficients that become an important and differentiating physical constant of a substance at the presumed low photon flux created by the cavitation process. The molar absorption coefficient, molar extinction coefficient, or molar absorptivity, is a measurement of how strongly a chemical species absorbs light at a given wavelength. It is an intrinsic property of the species; the actual absorbance, A, of a sample is dependent on the pathlength, [symbol] , and the concentration, C, of the species via the Beer-Lambert law. Thus absorption efficiency is directly measured through the absorption coefficient. The mass attenuation coefficient is a measurement of how strongly a chemical species or substance absorbs or scatters light at a given wavelength, per unit mass.

Porphyrins are a class of substances known to localize in tumors, have photodynamic sensitivity, and high extinction coefficients. These substances have been evaluated as sonosensitizers. This was studied on the presumption that sonoluminescence might cause electronic excitation of porphyrins by energy transfer and initiate a photochemical process, thus replicating the PDT process. Porphyrins or related molecules with high extinction coefficients appear to be those that are also sonosensitizers. In contrast to anti-cancer drugs, porphyrins and porphyrin-related molecules are nontoxic in the absence of ultrasound or light activating energy.

Porphyrins, the so-called “expanded porphyrins”, and related polypyrrole structures are members of a class of macrocycles capable of forming stable complexes with metals. The metal is constrained (as its cation) within a central binding cavity of the macrocycle (the “core”). The anions associated with the metal cation are found above and below the core; and are called apical ligands. Examples of this class of macrocycles are porphyrins, porphyrin isomers, porphyrin-like macrocycles, bcnzoporphyrins, texaphyrins, alaskaphyrins, sapphyrins, rubyrins, porphycenes, chlorins, benzochlorins, bacteriochlorins, and purpurins. Examples of metalized prophyrins are chlorophyll where the metal is Mg(II), Vitamin B12 where the metal is Co(II), and Heme where the metal is Fe(II).

Despite recent advances in PDT and SDT, there is still a need for additional sensitizers, including photosensitizers, sonosensitizers and dual acting (photo- and sono-) sensitizers for use in diagnostic and therapeutic applications. Sonosensitizers offer the greatest therapeutic possibilities because of their ability to act on deeply embedded tumors. Hematoporphyrin, a common photodynamic sensitizer, increased the killing of sarcoma in mice effectively, however, a percentage of malignant cells remained undamaged. The ability to enhance drug cytotoxicity with ultrasound and/or light that enables efficient but localized effects on a pathological site with minimal damage to peripheral healthy tissue is a valuable clinical asset that energy activated therapy appears to provide.

### BRIEF SUMMARY OF THE INVENTION

The invention is based on the unexpected discovery of novel sonosensitizers having optimized sonodynamic properties while also having photodynamic properties. The sensitizers have low toxicity prior to activation as determined in LC50 studies; target areas of tumor growth and activity including: tumor cells, tumor cell membranes, or the neovascular network of the tumor; are rapidly cleared from non-tumor compartments of the body; are highly sensitive to activation using readily available and safe ultrasound frequencies and intensities and red light; and have minimal side effects to the body systemically or to the local area upon activation. Accordingly, the present invention provides methods and compounds having a general formula I:

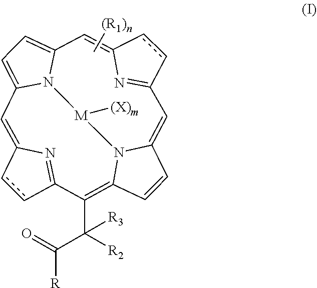

or its geometric isomers, enantiomers, diastereomers, racemates, pharmaceutically acceptable salts, prodrugs and solvates thereof, wherein R is O R₄ or R N₄R₅ each R₄ and R₅ is independently selected from hydrogen, a substituted or unsubstituted, saturated or unsaturated alkyl group or a substituted or unsubstituted aryl group or other blocking or protective group; alternatively, R₄ and R₅ can be taken together with the nitrogen they are attached to form a substituted or unsubstituted heterocyclic group; each R₁ is independently selected from a substituted or unsubstituted, saturated or unsaturated alkyl group, a substituted or unsubstituted aryl group, acid, ester, amide, amine, substituted amine, acyl, hydroxy, ether, halogen, nitrile, aldehyde, thiol, thioether, sulfonic acid, sulfonate, sulfonamide, and sulfate; R₂ and R₃ are independently selected from hydrogen, a substituted or unsubstituted, saturated or unsaturated alkyl group;
n is zero or an integer from 1 to 10;
each [symbol] is a single or double bond;
M represents a metal at oxidation state I-VII, preferably tin (Sn);
X is selected from the group consisting of anions, acids (acetate, for example) F, Cl, Br, I, H, CN, a substituted or unsubstituted hydroxide group, a substituted or unsubstituted amino group, a substituted or unsubstituted, straight or branched C1-C20 alkyl group, an acyl group, a thiolate group or a dialkylamino group; preferably OH and/or acetate are preferred and m represents 2, 3, 4 or 5 and is chosen to maintain the electric neutrality of the metal complex compound.

A preferred embodiment of the invention relates to chlorin derivatives, such as metallated chlorin-e6 (ce6) and derivatives thereof, including its ester or amide derivatives. These chlorin derivatives have sonosensitizing properties and may be used to treat diseases and other conditions in humans and animals. Moreover, the ce6 derivatives of the present invention may be modified, derivatized and/or conjugated to a delivery moiety to enhance the ability of the derivative to target predetermined cells or structures in vitro or in vivo.

The compositions (and/or their metabolites) of the present invention are activated by sound and/or light, exhibit substantial absorption in the therapeutic frequencies of ultrasound and/or red light; produce high cytotoxic component yield; can be produced in pure, monomeric form; may be derivatized, modified and/or conjugated to optimize properties of ultrasound activation and/or light activation, tissue biodistribution, and toxicity; and are rapidly cleared and excreted. They afford tumor targeting by covalent or physical attachment to cell membranes or penetrate into the cells to enhance sonotoxicity and phototoxicity (cytotoxicity upon activation).

The invention further provides compounds for use in SDT, PDT, Sonophotodynamic therapy (SPDT), Ultrasound activated therapy (USAT), diagnostic (photodynamic diagnostics) and therapeutic applications. In some embodiments, the compounds preferentially absorb into target tissue, including hyper-proliferative tissue. Diseases and conditions, which can be treated, include cancer, including tumors.

### BRIEF DESCRIPTION OF THE DRAWINGS

FIG. 1 illustrates a Lethality curve for ACT4211L.

FIG. 2 is a dose-response curve of ACT42111, cytotoxicity of human melanoma cancer cell. X axis represents ACT4211 concentration, Y axis represents the % Cell death when compared to the vehicle control. Each points represents mean+SE (n=6); and FIG. 6 shows the absorption spectrum for ACT4211.

FIG. 3 illustrates a Lethality curve for ACT4211.

FIG. 4 is a dose-response curve of ACT4211 cytotoxicity of human melanoma cancer cell. X axis represents ACT4211 concentration, Y axis represents the % Cell death when compared to the vehicle control. Each points represents mean+SE (n=6).

FIG. 5A represents standard curves used to calculate cell number for each condition wherein y=0.0124X+1620 was used for RFU>5730. X axis represents cell number, Y axis represents RFU measurement.

FIG. 5B represents standard curves used to calculate cell number for each condition wherein y=0.022X+397.74 was used for RFU<5730. X axis represents cell number, Y axis represents RFU measurement.

FIG. 6 shows the absorption spectrum for ACT4211.

### DETAILED DESCRIPTION OF THE PREFERRED EMBODIMENTS

In a some embodiments, the compounds of the present invention are compounds represented by formula (I) as illustrated above, or its geometric isomers, enantiomers, diastereomers, racemates, pharmaceutically acceptable salts, prodrugs and solvates.

In some embodiments, the compounds of the present invention are represented by formula (II) as illustrated below, or its geometric isomers, enantiomers, diastereomers, racemates, pharmaceutically acceptable salts, prodrugs and solvates thereof:

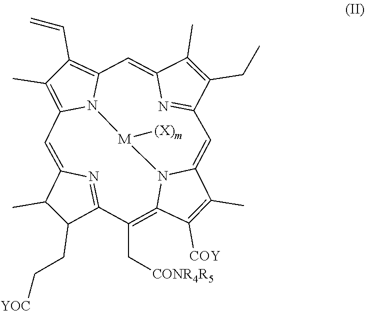

wherein Y is hydroxy, substituted hydroxy, prodrug group or an acceptable metal salt. In some embodiments NR₄R₅ are amino acid, amino acid derivative, or peptide. Amino acid derivatives are those derived from valine, leucine, isoleucine, threonine, methionine, phenylalanine, tryptophan, alanine, arginine, aspartic acid, cysteine, glutamic acid, glycine, histidine, proline, serine, tyrosine, asparagines, and glutamine. Amino acid-like derivatives including, but not limited to taurine, may also be used. Also useful are peptides, particularly those known to have affinity for specific receptors, including but not limited to, oxytocin, vasopressin, bradykinin, LHRH, thrombin, and the like. In some embodiments, NR₄R₅ represents an amine terminated polyethylene glycol (PEG). PEGylation provides for improved bioavailability, including longer circulation time and slower clearance. In particular, it improves the delivery of injectable proteins as well as other compounds. It can also be used for controlled agent release and optimized pharmacokinetics resulting in sustained duration. In addition, PEGylation may improve the safety profile with lower toxicity, immunogenicity, and antigenicity. It can also provide increased efficacy and decreased dosing frequency. PEGylation also improves agent solubility and stability and reduces susceptibility to proteolysis. The PEGs are selected from a broad range of molecular weights (5-60 kDa), functional groups, and attachment chemistries. For example, PEG can be 12-40 kDa, and can be branched or unbranched, binding can be through an —NHS reactive group, and binding sites can include lysine or histidine residues. In one example R₄ is hydrogen and R₅ is a —(CH₂CH₂O)ᵣCH₂CH₂OH wherein r is an integer between 1 and 100.

In some embodiments, the compounds of the present invention are compounds represented by formula (III) as illustrated below, or its geometric isomers, enantiomers, diastereomers, racemates, pharmaceutically acceptable salts, prodrugs and solvates thereof:

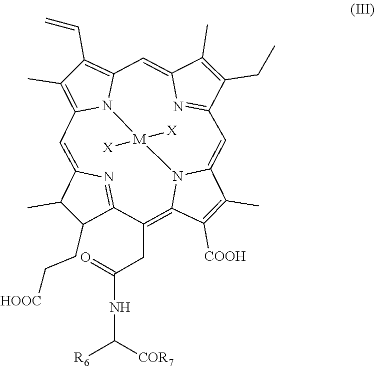

wherein R₆ is hydrogen, a substituted or unsubstituted, saturated or unsaturated alkyl, substituted or unsubstituted aryl; R₇ and is a hydroxy, substituted hydroxy, amine or substituted amine; M and X are as previously defined. In one embodiment, when M is at oxidation state IV, then m=2; some examples are Ti(IV), Zr(IV), Hf(IV), Sn(IV), Mo(IV), V(IV) and W(IV). When M is at oxidation state V, then m=3; some examples are V(V), Nb(V) and Ta(V). When M is at oxidation state VI, then m=4; some examples are Cr(VI), Mo(VI), W(VI) and Re(VI). When M is at oxidation state VII, then m=5; some examples are Tc(VII) and Re(VII). It is preferable to use metal complex compounds of general formula (I) with metals M at oxidation state IV. Preferably M at oxidation state IV includes Si(IV), Ti(IV), Sn(IV), Zr(IV), Hf(IV), Th(IV), Sn(IV), Mo(IV), V(IV) and W(IV). Sn(IV) are particularly preferred.

Compounds according to the invention include compounds of formula IV:

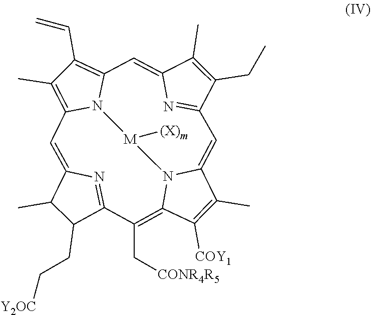

Wherein M, Y₁, Y₂, and NR₄R₅ are set forth in the Table A below:

### Tables A1-6

TABLE A1

| Cpd. | | | | |
|---|---|---|---|---|
| No. | M | Y₁ | Y₂ | —NR₄R₅ |
| 1 | Sn(IV) | OH | OH | —NHCH₂COOH |
| 2 | Sn(IV) | OMe | OH | —NHCH₂COOH |
| 3 | Sn(IV) | OH | OMe | —NHCH₂COOH |
| 4 | Sn(IV) | OEt | OH | —NHCH₂COOH |
| 5 | Sn(IV) | OH | OEt | —NHCH₂COOH |
| 6 | Sn(IV) | NH₂ | OH | —NHCH₂COOH |
| 7 | Sn(IV) | OH | NH₂ | —NHCH₂COOH |
| 8 | Sn(IV) | OH | OH | —NHCHCH₃COOH |
| 9 | Sn(IV) | OMe | OH | —NHCHCH₃COOH |
| 10 | Sn(IV) | OH | OMe | —NHCHCH₃COOH |
| 11 | Sn(IV) | OEt | OH | —NHCHCH₃COOH |
| 12 | Sn(IV) | OH | OEt | —NHCHCH₃COOH |
| 13 | Sn(IV) | NH₂ | OH | —NHCHCH₃COOH |
| 14 | Sn(IV) | OH | NH₂ | —NHCHCH₃COOH |
| 15 | Sn(IV) | OH | OH | —NHCHC₂H₅COOH |
| 16 | Sn(IV) | OMe | OH | —NHCHC₂H₅COOH |
| 17 | Sn(IV) | OH | OMe | —NHCHC₂H₅COOH |
| 18 | Sn(IV) | OEt | OH | —NHCHC₂H₅COOH |
| 19 | Sn(IV) | OH | OEt | —NHCHC₂H₅COOH |
| 20 | Sn(IV) | NH₂ | OH | —NHCHC₂H₅COOH |
| 21 | Sn(IV) | OH | NH₂ | —NHCHC₂H₅COOH |
| 22 | Sn(IV) | OH | OH | —NHCH(CHPh)COOH |
| 23 | Sn(IV) | OMe | OH | —NHCH(CHPh)COOH |
| 24 | Sn(IV) | OH | OMe | —NHCH(CHPh)COOH |
| 25 | Sn(IV) | OEt | OH | —NHCH(CHPh)COOH |
| 26 | Sn(IV) | OH | OEt | —NHCH(CHPh)COOH |
| 27 | Sn(IV) | NH₂ | OH | —NHCH(CHPh)COOH |
| 28 | Sn(IV) | OH | NH₂ | —NHCH(CHPh)COOH |
| 29 | Sn(IV) | OH | OH | —NHCH(CHOH)COOH |
| 30 | Sn(IV) | OMe | OH | —NHCH(CHOH)COOH |
| 31 | Sn(IV) | OH | OMe | —NHCH(CHOH)COOH |
| 32 | Sn(IV) | OEt | OH | —NHCH(CHOH)COOH |
| 33 | Sn(IV) | OH | OEt | —NHCH(CHOH)COOH |
| 34 | Sn(IV) | NH2 | OH | —NHCH(CHOH)COOH |
| 35 | Sn(IV) | OH | NH2 | —NHCH(CHOH)COOH |

TABLE A2

| Cpd. | | | | |
|---|---|---|---|---|
| No. | M | Y₁ | Y₂ | —NR₄R₅ |
| 36 | Sn(IV) | OH | OH | —NHCH(CH₂COOH)COOH |
| 37 | Sn(IV) | OMe | OFT | —NHCH(CH₂COOH)COOH |
| 38 | Sn(IV) | OH | OMe | —NHCH(CH₂COOH)COOH |
| 39 | Sn(IV) | OEt | OH | —NHCH(CH₂COOH)COOH |
| 40 | Sn(IV) | OH | OEt | —NHCH(CH₂COOH)COOH |
| 41 | Sn(IV) | NH₂ | OH | —NHCH(CH₂COOH)COOH |
| 42 | Sn(IV) | OH | NH₂ | —NHCH(CH₂COOH)COOH |
| 43 | Sn(IV) | OH | OH | —NHCH(CH₂CH₂COOH)COOH |
| 44 | Sn(IV) | OMe | OH | —NHCH(CH₂CH₂COOH)COOH |
| 45 | Sn(IV) | OH | OMe | —NHCH(CH₂CH₂COOH)COOH |
| 46 | Sn(IV) | OEt | OH | —NHCH(CH₂CH₂COOH)COOH |
| 47 | Sn(IV) | OH | OEt | —NHCH(CH₂CH₂COOH)COOH |
| 48 | Sn(IV) | NH₂ | OH | —NHCH(CH₂CH₂COOH)COOH |
| 49 | Sn(IV) | OH | NH₂ | —NHCH(CH₂CH₂COOH)COOH |
| 50 | Sn(IV) | OH | OH | —HNCH((CH₂)₄NH₂)COOH |
| 51 | Sn(IV) | OMe | OH | —HNCH((CH₂)₄NH₂)COOH |
| 52 | Sn(IV) | OH | OMe | —HNCH((CH₂)₄NH₂)COOH |
| 53 | Sn(IV) | OEt | OH | —HNCH((CH₂)₄NH₂)COOH |
| 54 | Sn(IV) | OH | OEt | —HNCH((CH₂)₄NH₂)COOH |
| 55 | Sn(IV) | NH₂ | OH | —HNCH((CH₂)₄NH₂)COOH |
| 56 | Sn(IV) | OH | NH₂ | —HNCH((CH₂)₄NH₂)COOH |
| 57 | Sn(IV) | OH | OH | —HNCH((CH₂)₃NH₂)COOH |
| 58 | Sn(IV) | OMe | OH | —HNCH((CH₂)₃NH₂)COOH |
| 59 | Sn(IV) | OH | OMe | —HNCH((CH₂)₃NH₂)COOH |
| 60 | Sn(IV) | OEt | OH | —HNCH((CH₂)₃NH₂)COOH |
| 61 | Sn(IV) | OH | OEt | —HNCH((CH₂)₃NH₂)COOH |
| 62 | Sn(IV) | NH₂ | OH | —HNCH((CH₂)₃NH₂)COOH |
| 63 | Sn(IV) | OH | NH₂ | —HNCH((CH₂)₃NH₂)COOH |
| 64 | Sn(IV) | OH | OH | —HNCH((CH₂)₃NH(CNH₂)═NH)COOH |
| 65 | Sn(IV) | OMe | OH | —HNCH((CH₂)₃NH(CNH₂)═NH)COOH |
| 66 | Sn(IV) | OH | OMe | —HNCH((CH₂)₃NH(CNH₂)═NH)COOH |
| 67 | Sn(IV) | OEt | OH | —HNCH((CH₂)₃NH(CNH₂)═NH)COOH |
| 68 | Sn(IV) | OH | OEt | —HNCH((CH₂)₃NH(CNH₂)═NH)COOH |
| 69 | Sn(IV) | NH₂ | OH | —HNCH((CH₂)₃NH(CNH₂)═NH)COOH |
| 70 | Sn(IV) | OH | NH₂ | —HNCH((CH₂)₃NH(CNH₂)═NH)COOH |

TABLE A3

| Cpd. | | | | |
|---|---|---|---|---|
| No. | M | Y₁ | Y₂ | —NR₄R₅ |
| 71 | Sn(IV) | OH | OH | —HNCH(CH₂CONH₂)COOH |
| 72 | Sn(IV) | OMe | OH | —HNCH(CH₂CONH₂)COOH |
| 73 | Sn(IV) | OH | OMe | —HNCH(CH₂CONH₂)COOH |
| 74 | Sn(IV) | OEt | OH | —HNCH(CH₂CONH₂)COOH |
| 75 | Sn(IV) | OH | OEt | —HNCH(CH₂CONH₂)COOH |
| 76 | Sn(IV) | NH₂ | OH | —HNCH(CH₂CONH₂)COOH |
| 77 | Sn(IV) | OH | NH₂ | —HNCH(CH₂CONH₂)COOH |
| 78 | Sn(IV) | OH | OH | —HNCH(CH₂CH₂CONH₂)COOH |
| 79 | Sn(IV) | OMe | OH | —HNCH(CH₂CH₂CONH₂)COOH |
| 80 | Sn(IV) | OH | OMe | —HNCH(CH₂CH₂CONH₂)COOH |
| 81 | Sn(IV) | OEt | OH | —HNCH(CH₂CH₂CONH₂)COOH |
| 82 | Sn(IV) | OH | OEt | —HNCH(CH₂CH₂CONH₂)COOH |
| 83 | Sn(IV) | NH₂ | OH | —HNCH(CH₂CH₂CONH₂)COOH |
| 84 | Sn(IV) | OH | OH | —(NHCH₂CO)₂OH |
| 85 | Sn(IV) | OH | OH | —(NHCH₂CO)₃OH |
| 86 | Sn(IV) | OH | OH | —(NHCH₂CO)₄OH |
| 87 | Sn(IV) | OH | OH | —(NHCH2CO)₅OH |
| 88 | Sn(IV) | OH | OH | —(NHCH2CO)₆OH |
| 89 | Sn(IV) | OH | OH | —(NHCHCH₃CO)₄OH |
| 90 | Sn(IV) | OH | OH | —(NHCH(CHOH)CO)₄OH |
| 91 | Sn(IV) | OH | OH | —(NHCH((CH₂)₃NH₂)CO)₂OH |
| 92 | Sn(IV) | OH | OH | —(NHCH((CH2)₃NH₂)CO)₃OH |
| 93 | Sn(IV) | OH | OH | —(NHCH((CH₂)₃NH₂)CO)₄OH |
| 94 | Sn(IV) | OH | OH | —(NHCH(CH₂CONH₂)CO)₂OH |
| 95 | Sn(IV) | OH | OH | —N(histidine) |
| 96 | Sn(IV) | OH | OH | —N(proline) |
| 97 | Sn(IV) | OH | OH | —NH(CH₂CH₂O)ₙOH |
| 98 | Sn(IV) | OH | OH | —N(folate) |
| 99 | Sn(IV) | OH | OMe | —NH(CH₂CH₂O)ₙOH |
| 100 | Sn(IV) | OMe | OH | —NH(CH₂CH₂O)ₙOH |
| 101 | Ti(IV) | OH | OH | —NHCH₂COOH |
| 102 | Ti(IV) | OMe | OH | —NHCH₂COOH |
| 103 | Ti(IV) | OH | OMe | —NHCH₂COOH |
| 104 | Ti(IV) | OEt | OH | —NHCH₂COOH |
| 105 | Ti(IV) | OH | OEt | —NHCH₂COOH |

TABLE A4

| Cpd. | | | | |
|---|---|---|---|---|
| No. | M | Y₁ | Y₂ | —NR₄R₅ |
| 106 | Ti(IV) | NH₂ | OH | —NHCH₂COOH |
| 107 | Ti(IV) | OH | NH₂ | —NHCH₂COOH |
| 108 | Ti(IV) | OH | OH | —NHCHCH₃COOH |
| 109 | Ti(IV) | OMe | OH | —NHCHCH₃COOH |
| 110 | Ti(IV) | OH | OMe | —NHCHCH₃COOH |
| 111 | Ti(IV) | OEt | OH | —NHCHCH₃COOH |
| 112 | Ti(IV) | OH | OEt | —NHCHCH₃COOH |
| 113 | Ti(IV) | NH₂ | OH | —NHCHCH₃COOH |
| 114 | Ti(IV) | OH | NH₂ | —NHCHCH₃COOH |
| 115 | Ti(IV) | OH | OH | —NHCHC₂H₅COOH |
| 116 | Ti(IV) | OMe | OH | —NHCHC₂H₅COOH |
| 117 | Ti(IV) | OH | OMe | —NHCHC₂H₅COOH |
| 118 | Ti(IV) | OEt | OH | —NHCHC₂H₅COOH |
| 119 | Ti(IV) | OH | OEt | —NHCHC₂H₅COOH |
| 120 | Ti(IV) | NH₂ | OH | —NHCHC₂H₅COOH |
| 121 | Ti(IV) | OH | NH₂ | —NHCHC₂H₅COOH |
| 122 | Ti(IV) | OH | OH | —NHCH(CHPh)COOH |
| 123 | Ti(IV) | OMe | OH | —NHCH(CHPh)COOH |
| 124 | Ti(IV) | OH | OMe | —NHCH(CHPh)COOH |
| 125 | Ti(IV) | OEt | OH | —NHCH(CHPh)COOH |
| 126 | Ti(IV) | OH | OEt | —NHCH(CHPh)COOH |
| 127 | Ti(IV) | NH2 | OH | —NHCH(CHPh)COOH |
| 128 | Ti(IV) | OH | NH₂ | —NHCH(CHPh)COOH |
| 129 | Ti(IV) | OH | OH | —NHCH(CHOH)COOH |
| 130 | Ti(IV) | OMe | OH | —NHCH(CHOH)COOH |
| 131 | Ti(IV) | OH | OMe | —NHCH(CHOH)COOH |
| 132 | Ti(IV) | OEt | OH | —NHCH(CHOH)COOH |
| 133 | Ti(IV) | OH | OEt | —NHCH(CHOH)COOH |
| 134 | Ti(IV) | NH₂ | OH | —NHCH(CHOH)COOH |
| 135 | Ti(IV) | OH | NH₂ | —NHCH(CHOH)COOH |
| 136 | Ti(IV) | OH | OH | —NHCH(CH₂COOH)COOH |
| 137 | Ti(IV) | OMe | OH | —NHCH(CH₂COOH)COOH |
| 138 | Ti(IV) | OH | OMe | —NHCH(CH₂COOH)COOH |
| 139 | Ti(IV) | OEt | OH | —NHCH(CH₂COOH)COOH |
| 140 | Ti(IV) | OH | OEt | —NHCH(CH₂COOH)COOH |

TABLE A5

| Cpd. | | | | |
|---|---|---|---|---|
| No. | M | Y₁ | Y₂ | —NR₄R₅ |
| 141 | Ti(IV) | NH₂ | OH | —NHCH(CH₂COOH)COOH |
| 142 | Ti(IV) | OH | NH₂ | —NHCH(CH₂COOH)COOH |
| 143 | Ti(IV) | OH | OH | —NHCH(CH₂CH₂COOH)COOH |
| 144 | Ti(IV) | OMe | OH | —NHCH(CH₂CH₂COOH)COOH |
| 145 | Ti(IV) | OH | OMe | —NHCH(CH₂CH₂COOH)COOH |
| 146 | Ti(IV) | OEt | OH | —NHCH(CH₂CH₂COOH)COOH |
| 147 | Ti(IV) | OH | OEt | —NHCH(CH₂CH₂COOH)COOH |
| 148 | Ti(IV) | NH₂ | OH | —NHCH(CH₂CH₂COOH)COOH |
| 149 | Ti(IV) | OH | NH₂ | —NHCH(CH₂CH₂COOH)COOH |
| 150 | Ti(IV) | OH | OH | —HNCH((CH₂)₄NH₂)COOH |
| 151 | Ti(IV) | OMe | OH | —HNCH((CH₂)₄NH₂)COOH |
| 152 | Ti(IV) | OH | OMe | —HNCH((CH₂)₄NH₂)COOH |
| 153 | Ti(IV) | OEt | OH | —HNCH((CH₂)₄NH₂)COOH |
| 154 | Ti(IV) | OH | OEt | —HNCH((CH₂)₄NH₂)COOH |
| 155 | Ti(IV) | NH₂ | OH | —HNCH((CH₂)₄NH₂)COOH |
| 156 | Ti(IV) | OH | NH₂ | —HNCH((CH₂)₄NH₂)COOH |
| 157 | Ti(IV) | OH | OH | —HNCH((CH₂)₃NH₂)COOH |
| 158 | Ti(IV) | OMe | OH | —HNCH((CH₂)₃NH₂)COOH |
| 159 | Ti(IV) | OH | OMe | —HNCH((CH₂)₃NH₂)COOH |
| 160 | Ti(IV) | OEt | OH | —HNCH((CH₂)₃NH₂)COOH |
| 161 | Ti(IV) | OH | OEt | —HNCH((CH₂)₃NH₂)COOH |
| 162 | Ti(IV) | NH₂ | OH | —HNCH((CH₂)₃NH₂)COOH |
| 163 | Ti(IV) | OH | NH₂ | —HNCH((CH₂)₃NH₂)COOH |
| 164 | Ti(IV) | OH | OH | —HNCH((CH₂)₃NH(CNH₂)═NH)COOH |
| 165 | Ti(IV) | OMe | OH | —HNCH((CH₂)₃NH(CNH₂)═NH)COOH |
| 166 | Ti(IV) | OH | OMe | —HNCH((CH₂)₃NH(CNH₂)═NH)COOH |
| 167 | Ti(IV) | OEt | OH | —HNCH((CH₂)₃NH(CNH₂)═NH)COOH |
| 168 | Ti(IV) | OH | OEt | —HNCH((CH₂)₃NH(CNH₂)═NH)COOH |
| 169 | Ti(IV) | NH₂ | OH | —HNCH((CH₂)₃NH(CNH₂)═NH)COOH |
| 170 | Ti(IV) | OH | NH₂ | —HNCH((CH₂)₃NH(CNH₂)═NH)COOH |
| 171 | Ti(IV) | OH | OH | —HNCH(CH₂CONH₂)COOH |
| 172 | Ti(IV) | OMe | OH | —HNCH(CH₂CONH₂)COOH |
| 173 | Ti(IV) | OH | OMe | —HNCH(CH₂CONH₂)COOH |
| 174 | Ti(IV) | OEt | OH | —HNCH(CH₂CONH₂)COOH |
| 175 | Ti(IV) | OH | OEt | —HNCH(CH₂CONH₂)COOH |

TABLE A6

| Cpd. | | | | |
|---|---|---|---|---|
| No. | M | Y₁ | Y₂ | —NR₄R₅ |
| 176 | Ti(IV) | NH₂ | OH | —HNCH(CH₂CONH₂)COOH |
| 177 | Ti(IV) | OH | NH₂ | —HNCH(CH₂CONH₂)COOH |
| 178 | Ti(IV) | OH | OH | —HNCH(CH₂CH₂CONH₂)COOH |
| 179 | Ti(IV) | OMe | OH | —HNCH(CH₂CH₂CONH₂)COOH |
| 180 | Ti(IV) | OH | OMe | —HNCH(CH₂CH₂CONH₂)COOH |
| 181 | Ti(IV) | OEt | OH | —HNCH(CH₂CH₂CONH₂)COOH |
| 182 | Ti(IV) | OH | OEt | —HNCH(CH₂CH₂CONH₂)COOH |
| 183 | Ti(IV) | NH₂ | OH | —HNCH(CH₂CH₂CONH₂)COOH |
| 184 | Ti(IV) | OH | OH | —(NHCH₂CO)₂OH |
| 185 | Ti(IV) | OH | OH | —(NHCH₂CO)₃OH |
| 186 | Ti(IV) | OH | OH | —(NHCH₂CO)₄OH |
| 187 | Ti(IV) | OH | OH | —(NHCH₂CO)₅OH |
| 188 | Ti(IV) | OH | OH | —(NHCH₂CO)₆OH |
| 189 | Ti(IV) | OH | OH | —(NHCHCH₃CO)₄OH |
| 190 | Ti(IV) | OH | OH | —(NHCH(CHOH)CO)₄OH |
| 191 | Ti(IV) | OH | OH | —(NHCH((CH₂)₃NH₂)CO)₂OH |
| 192 | Ti(IV) | OH | OH | —(NHCH((CH₂)₃NH₂)CO)₃OH |
| 193 | Ti(IV) | OH | OH | —(NHCH((CH₂)₃NH₂)CO)₄OH |
| 194 | Ti(IV) | OH | OH | —(NHCH(CH₂CONH₂)CO)₂OH |
| 195 | Ti(IV) | OH | OH | —N(histidine) |
| 196 | Ti(IV) | OH | OH | —N(proline) |
| 197 | Ti(IV) | OH | OH | —NH(CH₂CH₂O)ₙOH |
| 198 | Ti(IV) | OH | OH | —N(folate) |
| 199 | Ti(IV) | OH | OMe | —NH(CH₂CH₂O)ₙOH |
| 200 | Ti(IV) | OMe | OH | —NH(CH₂CH₂O)ₙOH |

Compounds according to the invention include compounds of formula:

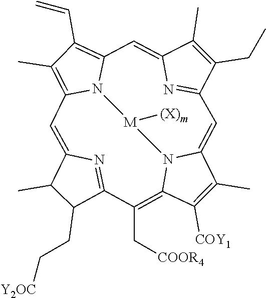

wherein M, Y₁, Y₂, and R₄ are set forth in the Table (B) below:

TABLE B

| Cpd. | | | | |
|---|---|---|---|---|
| No. | M | Y₁ | Y₂ | —R₄ |
| 1 | Sn(IV) | OH | OH | —H |
| 2 | Sn(IV) | OMe | OH | —H |
| 3 | Sn(IV) | OH | OMe | —H |
| 4 | Sn(IV) | OEt | OH | —H |
| 5 | Sn(IV) | OH | OEt | —H |
| 6 | Sn(IV) | NH₂ | OH | —H |
| 7 | Sn(IV) | OH | NH₂ | —H |
| 8 | Sn(IV) | OH | OH | —CH₃ |
| 9 | Sn(IV) | OMe | OH | —CH₃ |
| 10 | Sn(IV) | OH | OMe | —CH₃ |
| 11 | Sn(IV) | OEt | OH | —CH₃ |
| 12 | Sn(IV) | OH | OEt | —CH₃ |
| 13 | Sn(IV) | NH₂ | OH | —CH₃ |
| 14 | Sn(IV) | OH | NH₂ | —CH₃ |
| 15 | Sn(IV) | OH | OH | —C₂H₅ |
| 16 | Sn(IV) | OMe | OH | —C₂H₅ |
| 17 | Sn(IV) | OH | OMe | —C₂H₅ |
| 18 | Sn(IV) | OEt | OH | —C₂H₅ |
| 19 | Sn(IV) | OH | OEt | —C₂H₅ |
| 20 | Sn(IV) | NH₂ | OH | —C₂H₅ |
| 21 | Sn(IV) | OH | NH₂ | —C₂H₅ |
| 22 | Sn(IV) | OH | OH | —CH₂Ph |
| 23 | Sn(IV) | OMe | OH | —CH₂Ph |
| 24 | Sn(IV) | OH | OMe | —CH₂Ph |
| 25 | Sn(IV) | OEt | OH | —CH₂Ph |
| 26 | Sn(IV) | OH | OEt | —CH₂Ph |
| 27 | Sn(IV) | NH2 | OH | —CH₂Ph |
| 28 | Sn(IV) | OH | NH₂ | —CH₂Ph |

In one aspect an agent for use in treatment of a disease state or improving a condition associated with a disease state, the agent comprises one or more components selected from the group consisting of Sn(IV) chlorin e6 gly-gly amide dihydroxide sodium salt, Sn(IV) chlorin e6 gly-gly-gly-gly amide dihydroxide sodium salt, Sn(IV) chlorin e6 Taurine amide dihydroxide sodium salt, Sn(IV) chlorin e6 L-serine amide dihydroxide sodium salt, Sn(IV) chlorin e6 lycine amide dihydroxide sodium salt. In some embodiments two components are combined in a weight ratio between 10:1 and 1:10. In some embodiments three components are combined in a weight ratio between 10:1:1, 10:10:1, 1:10:1, 1:10:10 and 1:1:10. In another aspect an agent for use in treatment of a disease state or improving a condition associated with the disease, the agent comprising four components, the four components combined such that the greatest is present at not more than 70% by weight and the least is present at not less than 10% by weight. In some embodiments, the disease state is cancer, including tumors.

In some embodiments, the four components are selected from the group consisting of Sn(IV) chlorin c6 gly-gly amide dihydroxide sodium salt, Sn(IV) chlorin e6 gly-gly-gly-gly amide dihydroxide sodium salt, Sn(IV) chlorin e6 Taurine amide dihydroxide sodium salt, Sn(IV) chlorin e6 L-serine amide dihydroxide sodium salt. In some embodiments, the first component is Sn(IV) chlorin e6 gly-gly amide dihydroxide sodium salt, the second component is Sn(IV) chlorin e6 gly-gly-gly-gly amide dihydroxide sodium salt, the third component is Sn(IV) chlorin e6 Taurine amide dihydroxide sodium salt, the forth component is Sn(IV) chlorin e6 L-serine amide dihydroxide sodium salt.

In some embodiments, the four components are selected from the group consisting of Sn(IV) chlorin e6 gly-gly amide dihydroxide sodium salt, Sn(IV) chlorin e6 gly-gly-gly-gly amide dihydroxide sodium salt, Sn(IV) chlorin e6 Taurine amide dihydroxide sodium salt, Sn(IV) chlorin e6 L-serine amide dihydroxide sodium salt. In some embodiments, the first component is Sn(IV) chlorin e6 gly-gly amide dihydroxide sodium salt, the second component is Sn(IV) chlorin e6 gly-gly-gly-gly amide dihydroxide sodium salt, the third component is Sn(IV) chlorin e6 Taurine amide dihydroxide sodium salt, the forth component is Sn(IV) chlorin c6 lycine amide dihydroxide sodium salt.

In another aspect an agent for use in treatment of a disease state or improving a condition associated with the disease, the agent comprising five components, the five components arc combined such that the greatest is present at not more than 60% by weight and the least is present at not less than 10% by weight.

In some embodiments, the five components are selected from the group consisting of Sn(IV) chlorin e6 gly-gly amide dihydroxide sodium salt, Sn(IV) chlorin e6 gly-gly-gly gly amide dihydroxide sodium salt, Sn(IV) chlorin e6 Taurine amide dihydroxide sodium salt, Sn(IV) chlorin e6 L-serine amide dihydroxide sodium salt, Sn(IV) chlorin e6 lycine amide dihydroxide sodium salt. In some embodiments, the first component is Sn(IV) chlorin e6 gly-gly amide dihydroxide sodium salt, the second component is Sn(IV) chlorin e6 gly-gly-gly-gly amide dihydroxide sodium salt, the third component is Sn(IV) chlorin e6 Taurine amide dihydroxide sodium salt, the forth component is Sn(IV) chlorin e6 L-serine amide dihydroxide sodium salt, and the fifth component is Sn(IV) chlorin e6 lycine amide dihydroxide sodium salt.

The invention also provides a method for improving a condition associated with a disease state, using energy-activated therapy whereby the energy source is either ultrasound, light, or a combination of ultrasound or light, the method comprising administering an amount of an agent according to the invention to a mammal effective to improve one or more conditions of the mammal associated with a disease state or disease states.

In some embodiments the agent has a first component, a second component, a third component, a fourth component, and a fifth component in weight ratios such that the component present in the highest amount is present at 20-96% and the component present at the lowest amount is present at 1-20%. In some embodiments, the first component is Sn(IV) chlorin e6 gly-gly amide dihydroxide sodium salt, the second component is Sn(IV) chlorin e6 gly-gly-gly-gly amide dihydroxide sodium salt, the third component is Sn(IV) chlorin e6 Taurine amide dihydroxide sodium salt, the fourth component is Sn(IV) chlorin e6 L-serine amide dihydroxide sodium salt, and the fifth component is Sn(IV) chlorin e6 lycine amide dihydroxide sodium salt. In some embodiments the components are ground together to a soluble powder and administered to the mammal in that form. In some embodiments the components are dissolved in water and administered to the mammal in that form. In some embodiments the components are lyophilized to form an active ingredient part of the agent. In some embodiments the disease is cancer, including tumors. In some embodiments, the agent comprises a sonosensitizer. In some embodiments the agent comprises a photosensitizer. In some embodiments the agent comprises both a photosensitizer and a sonosensitizer.

Photodynamic therapy and sonodynamic therapy use agents to treat or ameliorate conditions associated with one or more disease states. Diseases states may include, for example, one or more types of cancer, including tumors. Particular agents that may be used include multiple component metal complexes. In some embodiments the metal complexes include tin. Some embodiments include chlorin e6. Some embodiments include aspartyl chlorin e6. In some embodiments a first metal complex is Sn(IV) chlorin e6 gly-gly amide dihydroxide sodium salt. In some embodiments, the second metal complex is Sn(IV) chlorin e6 gly-gly-gly-gly amide dihydroxide sodium salt. In some embodiments, the third metal complex is Sn(IV) chlorin e6 Taurine amide dihydroxide sodium salt. In some embodiments, the forth metal complex is Sn(IV) chlorin e6 L-serine amide dihydroxide sodium salt. In some embodiments first, second, third, and fourth metal complexes are combined into an agent in a weight ratio of approximately 4:2:1:1. Herein the term “approximately” includes values +/−2-10%.

In some embodiments a first metal complex is Sn(IV) chlorin e6 gly-gly amide dihydroxide sodium salt. In some embodiments, the second metal complex is Sn(IV) chlorin e6 gly-gly-gly-gly amide dihydroxide sodium salt. In some embodiments, the third metal complex is Sn(IV) chlorin e6 Taurine amide dihydroxide sodium salt. In some embodiments, the forth metal complex is Sn(IV) chlorin e6 lycine amide dihydroxide sodium salt. In some embodiments first, second, third, and fourth metal complexes are combined into an agent in a weight ratio of approximately 4:2:1:1. Herein the term “approximately” includes values +/−2-10%.

In some embodiments an agent for use in treatment of a disease state or improving a condition associated with the disease state, comprises one or more components selected from the group consisting of Sn(IV) chlorin e6 gly-gly amide dihydroxide sodium salt, Sn(IV) chlorin e6 gly-gly-gly-gly amide dihydroxide sodium salt, Sn(IV) chlorin e6 Taurine amide dihydroxide sodium salt, Sn(IV) chlorin e6 L-serine amide dihydroxide sodium salt, Sn(IV) chlorin e6 lycine amide dihydroxide sodium salt. In some embodiments comprising five components, each of the five components is combined in the agent in a weight ratio relation to the other four components between approximately 10:0:0:0:0 to approximately 0:10:10:10:10.

For example, one active composition comprising the following chemical components in the following weight ratios has been found to be effective in the treatment of various diseases in the body using ultrasound and/or light therapy. Each component is a good photodynamic agent. This is referred to in the Examples as ACT4211.

| | Weight |
|---|---|
| Component | Ratio |
| Sn(IV) chlorin e6 gly-gly amide dihydroxide sodium salt | 4 |
| Sn(IV) chlorin e6 gly-gly-gly-gly amide dihydroxide sodium salt | 2 |
| Sn(IV) chlorin e6 Taurine amide dihydroxide sodium salt | 1 |
| Sn (1V) chlorin e6 L-serine amide dihydroxide sodium salt | 1 |

In another example, an active composition comprising the following chemical components in the following weight ratios has been prepared and is to be tested in the treatment of various diseases in the body using ultrasound and/or light therapy. Each component is a good photodynamic agent. This is referred to in the Examples as ACT4211L.

| | Weight |
|---|---|
| Component | Ratio |
| Sn(IV) chlorin e6 gly-gly amide dihydroxide sodium salt | 4 |
| Sn(IV) chlorin e6 gly-gly-gly-gly amide dihydroxide sodium salt | 2 |
| Sn(IV) chlorin e6 Taurine amide dihydroxide sodium salt | 1 |
| Sn (1V) chlorin e6 lycine amide dihydroxide sodium salt | 1 |

In another example, an active composition comprising the following chemical components in the following weight ratios has been prepared to be tested in the treatment of various diseases in the body using ultrasound and/or light therapy. Each component is a good photodynamic agent.

| | Weight |
|---|---|
| Component | Ratio |
| Sn(IV) chlorin e6 gly-gly amide dihydroxide sodium salt | 4 |
| Sn(IV) chlorin e6 gly-gly-gly-gly amide dihydroxide sodium salt | 2 |
| Sn(IV) chlorin e6 Taurine amide dihydroxide sodium salt | 1 |
| Sn (1V) chlorin e6 L-serine amide dihydroxide sodium salt | 1 |
| Sn (1V) chlorin e6 lycine amide dihydroxide sodium salt | 1 |

There are discussions in the art of various types of metal complexes. For example, U.S. Pat. No. 4,656,186 mentions a serine free base compound, but not a tin chelate. U.S. Pat. Nos. 4,656,186, 4,675,338, 4,693,885; 4,977,177; 5,004,811; and 5,066,274 similarly mention preparation of metal complexes, but do not mention the tin chelates. U.S. Pat. No. 4,977,177 teaches attaching more than one amino acid to the tetrapyrrole at several sites. Each of the above references is herby incorporated by reference in its entirety.

In another aspect, the present invention provides a pharmaceutical composition comprising a compound of the invention and a pharmaceutically acceptable carrier.

The compounds of the present invention are formulated into final pharmaceutical compositions for administration to a subject or applied to an in vitro target using techniques well-known in the art, for example, as summarized in Remington's Pharmaceutical Sciences, Mack Publishing Co., Easton, Pa. The compounds of the invention are useful as photosensitizers, as sonosensitizers as therapeutic, and diagnostic agents, for example for treatment of several cancer types such as, but not limited to, melanoma, prostate, brain, colon, ovarian, breast, skin, lung, esophagus and bladder cancers and other hormone-sensitive tumors, as well as for treatment of age-related macular degeneration, and for killing cells, viruses, fungi and bacteria in samples and living tissues as well known in the art of PDT and other sonosensitizer applications.

The compounds of the invention arc useful, for example, in sensitizing neoplastic cells or other abnormal tissue to destruction by ultrasound frequencies. The wavelength of the ultrasound is preferably chosen to match the maximum absorbance of the sonosensitizer. The suitable energy for any of the compounds can readily be determined empirically but can be between 20 KHz to 20 MHz, intensity of 0.1 to 500 W/cm2 and duration of 0.5 sec. to 5 hours. In an alternative embodiment, the compounds can be activated by light waves, as typically employed in photodynamic therapy. In an alternative embodiment, the compounds can be activated by a combination of light waves and ultrasound.

The conjugation of proteins, e.g., hormones, growth factors or their derivatives, antibodies, peptides that bind specifically to target cells receptors, and of cell nutrients, e.g. tyrosine, can increase their retention in tumor and treated sites.

The invention further relates to a method of sonodynamic therapy, which comprises administering to a subject a therapeutically effective amount of a compound of the invention, followed by local ultrasound.

The compounds of the invention are also useful for sonodestruction of normal or malignant animal cells, as desired. Thus, the invention further provides the use of the compounds of the invention for in vivo, ex-vivo or in vitro killing of cells or infectious agents such as bacteria, viruses, parasites and fungi in a biological product, e.g. blood, Use of the compounds accordingly comprises treating the infected sample with the compound followed by ultrasound and/or red light irradiation of the sample. The present invention provides for the use of one or more compounds of the invention in the manufacture of a medicament for the treatment of cancer.

In some embodiments, the present invention includes the use of one or more compounds of the invention in the manufacture of a medicament that prevents further aberrant proliferation, differentiation, or survival of cells. For example, compounds of the invention may be useful in preventing tumors from increasing in size or from reaching a metastatic state. The subject compounds may be administered to halt or inhibit the progression or advancement of cancer or to induce tumor necrosis, tumor apoptosis, or inhibit tumor angiogenesis.

In addition, the instant invention includes use of the subject compounds to prevent a recurrence of cancer. This is accomplished in part because the use of the compounds in a therapeutic mode creates an inflammatory reaction and/or response that yields a “vaccine effect.”

This invention further embraces the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue.

Combination therapy includes the administration of the subject compounds in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, preferably compounds that are able to enhance the effect of the compounds of the invention. The compounds of the invention can be administered simultaneously (as a single preparation or separate preparation) or sequentially to the other drug therapy. In general, a combination therapy envisions administration of two or more drugs during a single cycle or course of therapy.

Alternatively or additionally, administration of the subject compounds can be staggered, thereby resulting in varied compartmental distribution.

The compounds are administered prior to activation by ultrasound or phototherapy. Preferably the compounds are administered at least one day before activation, generally between 2 and 5 days before activation, or between 24 and 96 hours before activation. In one aspect of the invention, the subject compounds may be administered in combination with one or more separate agents that modulate protein kinases involved in various disease states. Examples of such kinases may include, but are not limited to: serine/threonine specific kinases, receptor tyrosine specific kinases and non-receptor tyrosine specific kinases. Serine/threonine kinases include mitogen activated protein kinases (MAPK), meiosis specific kinase (Aurora), RAF and Aurora kinase. Examples of receptor kinase families include epidermal growth factor receptor (EGFR) (e.g. HER2/neu, HER3, HER4, ErbB, ErbB2, ErbB3, ErbB4, Xmrk, DER, Let23); fibroblast growth factor (FGF) receptor (e.g. FGF-R1, GFF-R2/BEK/CEK3, FGF-R3/CEK2, FGF-R4/TKF, KGF R); hepatocyte growth/scatter factor receptor (HGFR) (e.g, MET, RON, SEA, SEX); insulin receptor (e.g. IGFI-R); Eph (e.g. CEK5, CEK8, EBK, ECK, EEK, EHK-1, EHK-2, ELK, EPH, ERK, HEK, MDK2, MDK5, SEK); Axl (e.g. Mer/Nyk, Rse); RET; and platelet-derived growth factor receptor (PDGFR) (e.g. PDGFα-R, PDGβ-R, CSF1-R/FMS, SCF-R/C-KIT, VEGF-R/FLT, NEK/FLK1, FLT3/FLK2/STK-1). Non-receptor tyrosine kinase families include, but are not limited to, BCR-ABL (e.g. p43abl, ARG); BTK (e.g. ITK/EMT, TEC); CSK, FAK, FPS, JAK, SRC, BMX, FER, CDK and SYK.

In certain embodiments, the compounds of the invention are administered in combination with a known chemotherapeutic agent.

In certain embodiments, the compounds of the invention are administered in combination with a chemoprotective agent. Chemoprotective agents act to protect the body or minimize the side effects of chemotherapy. Examples of such agents include, but are not limited to, amfostine, mesna, and dexrazoxane.

In some embodiments of the invention, the subject compounds are administered in combination with radiation therapy. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

It will be appreciated that compounds of the invention can be used in combination with an immunotherapeutic agent. One form of immunotherapy is the generation of an active systemic tumor-specific immune response of host origin by administering a vaccine composition at a site distant from the tumor. Various types of vaccines have been proposed, including isolated tumor-antigen vaccines and anti-idiotype vaccines. Another approach is to use tumor cells from the subject to be treated, or a derivative of such cells (reviewed by Schirrmacher et al. (1995) J. Cancer Res. Clin. Oncol. 121:487). In U.S. Pat. No. 5,484,596, Hanna Jr. et al. claims a method for treating a resectable carcinoma to prevent recurrence or metastases, comprising surgically removing the tumor, dispersing the cells with collagenase, irradiating the cells, and vaccinating the patient with at least three consecutive doses of about 10⁷ cells.

It will be appreciated that the compounds of the invention may advantageously be used in conjunction with one or more known adjunctive therapeutic agents.

In some embodiments, compounds of the invention can be used to induce or inhibit apoptosis, a physiological cell death process critical for normal development and homeostasis. Alterations of apoptotic pathways contribute to the pathogenesis of a variety of human diseases. Compounds of the invention, as modulators of apoptosis, will be useful in the treatment of a variety of human diseases with aberrations in apoptosis including cancer (particularly, but not limited to, follicular lymphomas, carcinomas with p53 mutations, hormone dependent tumors of the breast, prostate and ovary, and precancerous lesions such as familial adenomatous polyposis).

### DEFINITIONS

Listed below are definitions of various terms used to describe this invention. Unless a specific meaning is stated for a term used herein, it is intended that the term be given its usual meaning in the art.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, aminocarbonylcycloalkyl, aminocarbonylheterocyclyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid sulfonic acid, sulfonyl, phosphonic acid, aryl, heteroaryl, heterocyclic, and aliphatic. It is understood that the substituent may be further substituted.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.”

The phrase “adjunctive therapy” encompasses treatment of a subject with agents that reduce or avoid side effects associated with the combination therapy of the present invention, including, but not limited to, those agents, for example, that reduce the toxic effect of anticancer drugs, e.g., bone resorption inhibitors, cardioprotective agents; prevent or reduce the incidence of nausea and vomiting associated with chemotherapy, radiotherapy or surgical procedures; or reduce the incidence of infection associated with the administration of myelosuppressive anticancer drugs.

The term “effective amount of the subject compounds,” with respect to the subject method of treatment, refers to an amount of the subject compound which, when delivered as part of desired dose regimen, brings about, e.g. a change in the rate of cell proliferation and/or state of differentiation and/or rate of survival of a cell to clinically acceptable standards. This amount may further relieve to some extent one or more of the symptoms of a neoplasia disorder, including, but is not limited to: 1) reduction in the number of cancer cells; 2) reduction in tumor size; 3) inhibition (i.e., slowing to some extent, preferably stopping) of cancer cell infiltration into peripheral organs; 4) inhibition (i.e., slowing to some extent, preferably stopping) of tumor metastasis; 5) inhibition, to some extent, of tumor growth; 6) relieving or reducing to some extent one or more of the symptoms associated with the disorder; and/or 7) relieving or reducing the side effects associated with the administration of anticancer agents.

The term “inhibition,” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention.

The phrase a “radio therapeutic agent” refers to the use of electromagnetic or particulate radiation in the treatment of neoplasia.

The term “recurrence” as used herein refers to the return of cancer after a period of remission. This may be due to incomplete removal of cells from the initial cancer and may occur locally (the same site of initial cancer), regionally (in vicinity of initial cancer, possibly in the lymph nodes or tissue), and/or distally as a result of metastasis.

The term “treatment” refers to any process, action, application, therapy, or the like, wherein a mammal, including a human being, is subject to medical aid with the object of improving the mammal's condition, directly or indirectly.

By a “therapeutically effective amount” of a compound of the invention is meant an amount of the compound that confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.01 mg/Kg to about 500 mg/Kg, preferably from about 0.1 to about 10 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). As used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound that is convertible in vivo by metabolic means (e.g. by hydrolysis) to a compound of the invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard [symbol] Larsen, et al., (ed). “Design and Application of Prodrugs, Textbook of Drug Design and Development”, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

The term, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers arc described in the most recent edition of Remington's Pharmaceutical Sciences, a standard reference text in the field, which is incorporated herein by reference. The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this invention may be modified by appending appropriate functional groups and/or moieties to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques that are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers and/or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

### Pharmaceutical Compositions

The pharmaceutical compositions of the present invention comprise a therapeutically effective amount of a compound or compounds. In some embodiments, the active ingredients and mixtures of active ingredients may be used, for example, in pharmaceutical compositions comprising a pharmaceutically acceptable carrier prepared for storage and subsequent administration. Also, some embodiments include use of the above-described active ingredients with a pharmaceutically acceptable carrier or diluent. Acceptable carriers or diluents for therapeutic use are well known in the pharmaceutical art, and are described, for example, in Remington's Pharmaceutical Sciences, 18th Ed., Mack Publishing Co., Easton, PA (1990).

### Methods of Administration

The pharmaceutical compositions of this invention may be administered sublingually, orally, parenterally, by inhalation spray, topically, rectally, nasally, buccally, vaginally or via an implanted reservoir, preferably by oral administration or administration by injection, as deemed appropriate by those of skill in the art for bringing the compositions of the invention into optimal contact with the target tissue.

### EXAMPLES

The compounds of the present invention will be better understood in connection with the following non-limiting examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the claims. Compounds of the invention can be prepared by procedures known to those skilled in the art, such as U.S. Pat. No. 6,462,192 that is hereby incorporated by reference by its entirety.

### Example 1. Treatment of Skin Cancer

As an example, consider photodynamic therapy as a treatment for basal cell carcinoma. Basal cell carcinoma is the most common form of skin cancer in humans. Conventional treatment of basal cell carcinoma involves surgical excision, cryogenic treatment with liquid nitrogen, or localized chemotherapy with 5-fluorouracil or other agents. Applying a photosensitizer precursor (aminolevulinic acid or methyl aminolevulinate). A waiting period of a few hours is allowed to elapse, during which time aminolevulinic acid will be taken up by cells, and aminolevulinic acid will be converted by the cells to protoprophyrin IX, a photosensitizer.

The physician shines a bright red light (from an array of light-emitting diodes or a diode laser) on the area to be treated. The light exposure lasts a few minutes to a few tens of minutes. Protoprophyrin IX absorbs light, exciting it to an excited singlet state.

Intersystem crossing occurs, resulting in excited triplet protoprophyrin IX. Energy is transferred from triplet protoporphyrin IX to triplet oxygen, resulting in singlet (ground state) protoporphyrin IX and excited singlet oxygen. Singlet oxygen reacts with biomolecules, fatally damaging some cells in the treatment area. Within a few days, the exposed skin and carcinoma will scab over and flake away. In a few weeks, the treated area has healed, leaving healthy skin behind. For extensive malignancies, repeat treatments may be required. It is also common to experience pain from the area treated. After the treatment the patient will need to avoid excessive exposure to sunlight for a period of time.

### Example 2. Toxicity of ACT4211L

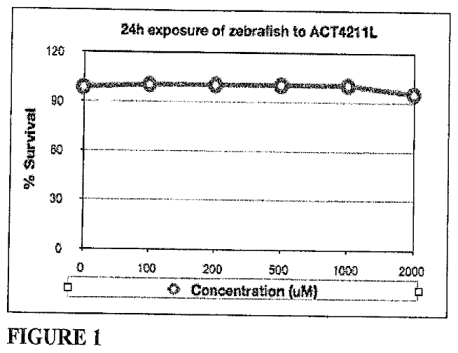

LC50 determination was carried out as described in Lewis, Thomas J. “Toxicity and cytopathogenic properties toward human melanoma cells of activated cancer therapeutics in zebra fish.” Integrative Cancer Therapies 9.1 (2010): 84-92. Briefly, 20 hpf zebrafish (n=30) were treated with ACT4211L at: 100, 200, 300, 400, 500, 600, 750, 850, 1000, 1500 and 2000 μM for 28 hours at 28° C. and lethality was recorded at 48 hpf. Significant lethality was not observed (FIG. 1). No significant lethality was observed in the repeat experiment. No further testing was performed.

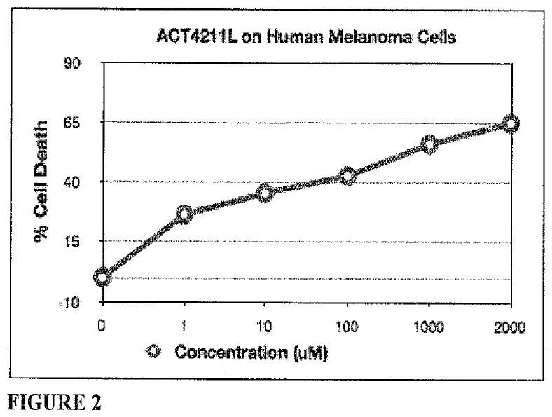

Assessment of cytotoxicity for melanoma cancer cell line WM-266-4. ACT4211L exhibited significant cytotoxic effect on human melanoma cancer cells WM-266-4 in vitro. A dose response effect was observed (FIG. 2); 5.5, 42.7, 56.0, and 64.8% cell death was observed at: 1, 10, 100 and 1000 μM concentration, respectively.

TABLE I
Results of LC50 determination.

| | # | | # | | | |
|---|---|---|---|---|---|---|
| Concen- | Dead | | Dead | | Mean | |
| tration | Fish | Mortality | Fish | Mortality | Mortality | Survival |
| (μM) | (exp 1) | (%) | (exp 2) | (%) | (%) | (%) |
| 0 | 1 | 3.3 | 0 | 0.0 | 1.7 | 98.3 |
| 100 | 0 | 0.0 | 0 | 0.0 | 0.0 | 100.0 |
| 200 | 0 | 0.0 | 0 | 0.0 | 0.0 | 100.0 |
| 300 | 0 | 0.0 | 0 | 0.0 | 0.0 | 100.0 |
| 400 | 1 | 3.3 | 0 | 0.0 | 1.7 | 98.3 |
| 500 | 0 | 0.0 | 0 | 0.0 | 0.0 | 100.0 |
| 600 | 1 | 3.3 | 0 | 0.0 | 1.7 | 98.3 |
| 750 | 0 | 0.0 | 0 | 0.0 | 0.0 | 100.0 |
| 850 | 0 | 0.0 | 0 | 0.0 | 0.0 | 100.0 |
| 1000 | 0 | 0.0 | 0 | 0.0 | 0.0 | 100.0 |
| 1500 | 0 | 0.0 | 0 | 0.0 | 0.0 | 100.0 |
| 2000 | 3 | 10.0 | 0 | 0.0 | 5.0 | 95.0 |

TABLE II
One-way analysis of variance (One-way ANOVA)
P value 0.0001
Are means signif. different? (P < 0.05) Yes
Number of groups 6

| Dunnett's Multiple | Mean | | | |
|---|---|---|---|---|
| Comparison Test | Diff. | q | P value | 95% CI of diff |
| 0 vs 0.1 | 7849 | 2.185 | P > 0.05 | −1706 to 17400 |
| 0 vs 1 | 10570 | 2.942 | P < 0.05 | 1015 to 20120 |
| 0 vs 10 | 12740 | 3.547 | P < 0.01 | 3186 to 22300 |
| 0 vs 100 | 16680 | 4.644 | P < 0.01 | 7126 to 26240 |
| 0 vs 1000 | 19300 | 5.373 | P < 0.01 | 9745 to 28850 |

TABLE III
Results of cytotoxicity assessment for human
melanoma cancer cell WM-266-4

| | | | % | % of | SD | | SE%/ |
|---|---|---|---|---|---|---|---|
| ACT4211L | | | of | Cell | % of | | of |
| (μM) | Mean | SD | Control | Death | Control | SE | Control |
| 0 | 29806 | 7194 | 100.0 | 0.0 | 24.1 | 2936 | 9.9 |
| 0.1 | 21958 | 9590 | 73.7 | 26.3 | 32.2 | 3914 | 1.1 |
| 1 | 19237 | 5279 | 64.5 | 35.5 | 17.7 | 2155 | 7.2 |
| 10 | 27066 | 7482 | 57.3 | 42.7 | 25.1 | 3054 | 10.2 |
| 100 | 13125 | 1938 | 44.0 | 56.0 | 6.5 | 791 | 2.7 |
| 1000 | 10506 | 956 | 35.2 | 64.8 | 3.2 | 390 | 1.3 |

### Example 3. Toxicity of Compound ACT4211

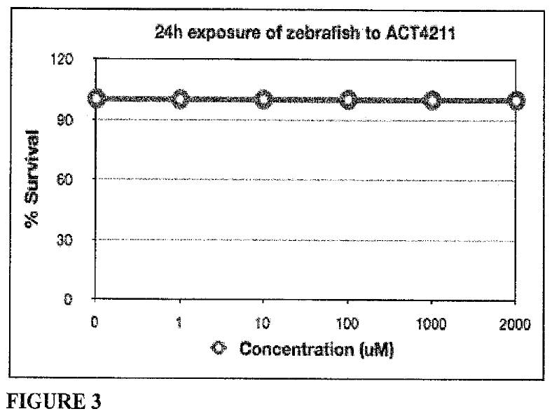

LC50 determination was made as described in Lewis, Thomas J. “Toxicity and cytopathogenic properties toward human melanoma cells of activated cancer therapeutics in zebra fish.” Integrative Cancer Therapies 9.1 (2010): 84-92. Briefly, 20 hpf zebrafish (n=30) were treated with ACT4211 at: 1, 10, 100, 1000 and 2000 μM for 28 hours at 28° C. and lethality was recorded at 48 hpf. No lethality was observed up to 2000 μM (FIG. 3). No further testing was performed.

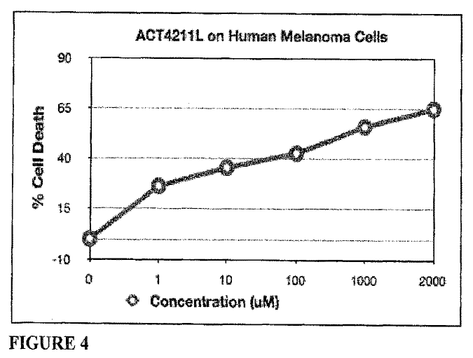

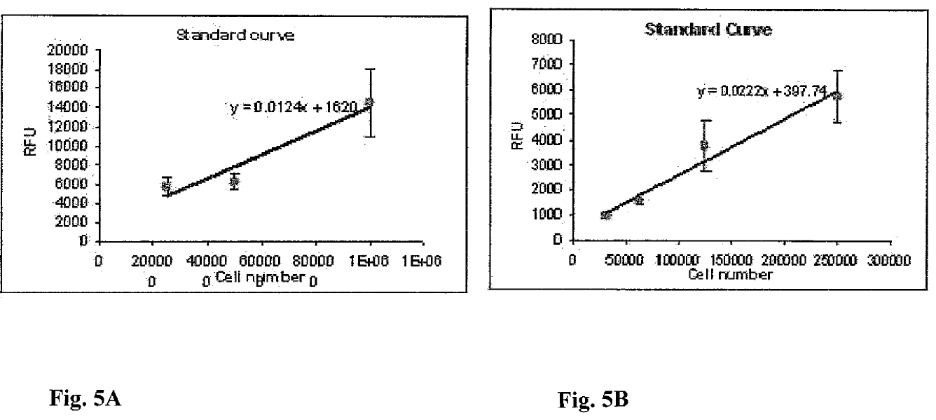

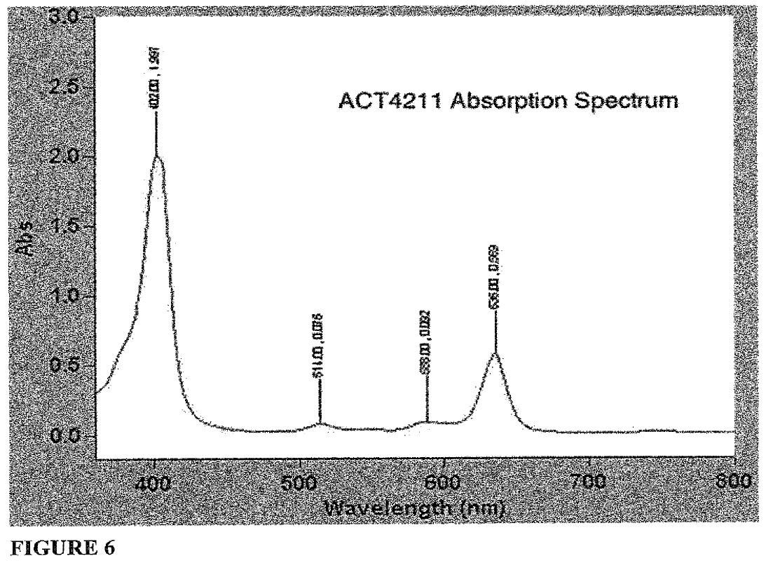

Assessment of ACT4211 cytotoxicity for melanoma cancer cell line WM-266-4: ACT4211exhibited significant cytotoxic effect on human melanoma cancer cells WM-266-4 in vitro. A dose response effect was observed; 57%, 81%, and 87% cell death was observed at: 100, 1000 and 2000 μM concentration, respectively (FIG. 4).

TABLE IV
Results of LC50 determination-ACT4211

| | # | | # | | Mean | |
|---|---|---|---|---|---|---|
| Concen- | Dead | Mor- | Dead | Mor- | Mor- | |
| tration | Fish | tality | Fish | tality | tality | Survival |
| (μM) | (exp 1) | (%) | (exp 2) | (%) | (%) | (%) |
| 0 | 0 | 3.3 | 0 | 0.0 | 0.0 | 100.0 |
| 1 | 0 | 0.0 | 0 | 0.0 | 0.0 | 100.0 |
| 10 | 0 | 0.0 | 0 | 0.0 | 0.0 | 100.0 |
| 100 | 0 | 0.0 | 0 | 0.0 | 0.0 | 100.0 |
| 1000 | 0 | 0.0 | 0 | 0.0 | 0.0 | 100.0 |
| 2000 | 0 | 0.0 | 0 | 0.0 | 0.0 | 100.0 |

TABLE V
One-way analysis of variance (One-way ANOVA)
P value <0.0001
Are means signif. different? (P < 0.05) Yes
Number of groups 6

| Dunnett's Multiple | Mean | | | |
|---|---|---|---|---|
| Comparison Test | Diff. | q | P value | 95% CI of diff |
| 0 vs 1 | 1641 | 2.565 | P > 0.05 | −60.52 to 3343 |
| 0 vs 10 | 69.17 | 0.1081 | P > 0.05 | −1633 to 1771 |
| 0 vs 100 | 2330 | 3.642 | P < 0.01 | 628.0 to 4032 |
| 0 vs 1000 | 4119 | 6.438 | P < 0.01 | 2417 to 5821 |
| 0 vs 2000 | 4631 | 7.238 | P < 0.01 | 2929 to 6333 |

TABLE VI
Results of cytotoxicity assessment for human melanoma cancer
cell WM-266-4

| ACT4211 | Mean | SD | % of | | |
|---|---|---|---|---|---|
| Conc. (uM) | (cell #) | (Cell #) | cell death | SD(%) | SE (%) |
| 0 | 344034 | 79063 | 0 | 23 | 9.4 |
| 1 | 359326 | 111434 | −4 | 32 | 13.2 |
| 10 | 335549 | 78258 | 2 | 23 | 9.3 |
| 100 | 147444 | 24512 | 57 | 7 | 2.9 |
| 1000 | 66851 | 9354 | 81 | 3 | 1.1 |
| 2000 | 43788 | 10132 | 87 | 3 | 1.2 |

TABLE VI
Results of cytotoxicity assessment for human melanoma cancer
cell WM-266-4

| ACT4211 | Mean | SD | % of | | |
|---|---|---|---|---|---|
| Conc. (uM) | (cell #) | (Cell #) | cell death | SD(%) | SE (%) |
| 0 | 344034 | 79063 | 0 | 23 | 9.4 |
| 1 | 359326 | 111434 | −4 | 32 | 13.2 |
| 10 | 335549 | 78258 | 2 | 23 | 9.3 |
| 100 | 147444 | 24512 | 57 | 7 | 2.9 |
| 1000 | 66851 | 9354 | 81 | 3 | 1.1 |
| 2000 | 43788 | 10132 | 87 | 3 | 1.2 |

### Example 4. Studies of Compound ACT4211

In this study the effect of SDT with ACT4211 on S-180 sarcoma in mice was examined, as described in Wang, Xiaohuai, Thomas J. Lewis, and Doug Mitchell. “The tumoricidal effect of sonodynamic therapy (SDT) on S-180 sarcoma in mice.” Integrative cancer therapies 7.2 (2008): 96-102. Tumor growth inhibition was visible even when covered by barrier of bone. Pathological slices showed coagulated necrosis or metamorphic tissue with inflammatory reaction in the tumor taken from 2 hours to 36 hours after SDT. These data revealed that SDT with ACT4211 inhibited growth of mouse S-180 sarcoma and the inhibitive effect was sound intensity dependent. SDT also induced some inflammation while it destroyed the tumor, indicative of a “vaccine” affect. ACT4211 shows great promise for clinical use in the future.

TABLE 1
Tumor weight in each group 15 days after treatment

| Group | Mean of tumor weight (g) | P (Comparing with C) |
|---|---|---|
| C | 0.361 ± 0.094 | |
| U | 0.440 ± 0.275 | >0.05 |
| S | 0.272 ± 0.328 | >0.05 |
| SU | 0.009 ± 0.003 | <0.01 |

Comparing with group C (control), the tumor weight in group SU was significantly lower (P<0.01). The tumor weight in group U and S had no significant difference like that of group C. This demonstrated that the ACT4211 plus sound treatment inhibited S-180 sarcoma in mice.

TABLE 2
Tumor size in each group 15 days after treatment

| Group | Mean of tumor size (cm³) | P (Comparing with C) |
|---|---|---|
| C | 0.865 ± 0.124 | |
| U | 0.799 ± 0.315 | >0.05 |
| S | 0.611 ± 0.190 | >0.05 |
| SU | 0.047 ± 0.019 | <0.01 |

As Table 3 shows, the tumor weight in the three SDT treated groups was much lower than that in Control group (P<0.05). These results conform to the conclusion that SDT with ACT4211 inhibits S-180 sarcoma in mice. It is very clear that the higher intensity of ultrasound used, the higher inhibitive response was produced at the range of ultrasound intensity from 0.3 W/cm2 to 1.2 W/cm2.

TABLE 3
Tumor weight in each group 15 days after treatment

| | Mean of tumor | P (Comparing | P (Comparing |
|---|---|---|---|
| Group | weight (g) | with Control) | with SU1) |
| Control | 0.361 ± 0.094 | | <0.05 |
| SU1 | 0.0425 ± 0.025 | <0.05 | |
| SU2 | 0.021 ± 0.006 | <0.01 | <0.05 |
| SU3 | 0.009 ± 0.003 | <0.01 | <0.01 |

Tumor size was measured with sliding calipers every one or two days. The results are shown in Table 4.

TABLE 4
the tumor size in each group 15 days after treatment

| | Mean of tumor | P(Comparing |
|---|---|---|
| Group | size (cm³) | with SU1) |
| Control | 0.865 ± 0.124 | <0.05 |
| SU1 | 0.383 ± 0.113 | |
| SU2 | 0.118 ± 0.020 | <0.05 |
| SU3 | 0.047 ± 0.019 | <0.01 |

As Table 4 shows, the tumors in the 3 SDT treatment groups were much smaller than that in Control group (P<0.05). The inhibitive effect of SDT with ACT4211 was sound intensity dependent.

The pathological study results in group SU (ACT4211 20 mg/Kg and ultrasound of 1.2 W/cm2) also showed superior results. Pathological slices were made from the mice sacrificed at 2 hours, 36 hours and 15 days after SDT. Coagulated necrosis or metamorphic tissue with inflammatory reaction in the tumor was observed and that the processes of necroses, degeneration and inflammation were further enhanced 36 hours after the SDT treatment. Fifteen days after SDT, only coagulated necroses and vacuole degeneration was visible in the tumor, but no living tumor cells could be identified. There was some inflammation and fibrosis around the necrotic or degenerative tumor. These data revealed that SDT with ACT4211 destroyed the S-180 sarcoma mouse very rapidly. The degeneration of tumor induced by SDT occurred almost immediately or at least within two hours after SDT treatment. These data also revealed that along with the necroses and degeneration of the tumor, SDT also induced inflammatory reaction in the tumor and the reaction may last for seven days. Observations with confocal laser scanning microscopy suggest that ACT4211 accumulates specifically within tumor cells.

SDT with a piece of bone between tumor and ultrasound was performed as described in Wang, Xiaohuai, Thomas J. Lewis, and Doug Mitchell. “The tumoricidal effect of sonodynamic therapy (SDT) on S-180 sarcoma in mice.” Integrative cancer therapies 7.2 (2008): 96-102. The results are shown in Table 5.

TABLE 5
Tumor weight in each group 15 days after treatment

| | Mean of tumor | P(Comparing |
|---|---|---|
| Group | weight (g) | with C) |
| C | 0.73466 ± 0.0781 | |
| SU | 0.07416 ± 0.0158 | >0.01 |

SDT with a piece of bone between tumor and ultrasound was still able to inhibit the tumor growth. This revealed that 1 MHz ultrasound can pass through bone, activate the sensitizer in the tumor and lead to tumor destruction.

The inhibitive effect of SDT with different ultrasound frequency on S-180 sarcoma in mice Was carried out as described in Wang, Xiaohuai, Thomas J. Lewis, and Doug Mitchell. “The tumoricidal effect of sonodynamic therapy (SDT) on S-180 sarcoma in mice.” Integrative cancer therapies 7.2 (2008): 96-102. The results are shown in Table 6.

TABLE 6
Tumor weight in each group 8 days after treatment

| | Mean of tumor | P (Comparing | P(Comparing |
|---|---|---|---|
| Group | weight (g) | with C) | with SU2) |
| C | 0.43208 ± 0.128413 | | |
| SU1 | 0.12515 ± 0.019856 | <0.01 | >0.05 |
| SU2 | 0.111967 ± 0.031018 | <0.01 | |
| SU3 | 0.121633 ± 0.020449 | <0.01 | >0.05 |

Compared to group C, the tumor weight in every SDT treated groups was significantly lower (P<0.01). This demonstrated again that the ACT4211 plus sound treatment did inhibit S-180 sarcoma in mice. The data suggests that 0.5 to 2.5 MHz ultrasounds were all able to active ACT4211 and destroy the tumor.

### Example 5. Sono-Photodynamic Therapy

A dose of 45 mg of photo/sonosensitizer is administered sublingually over 2 to 5 hours. No photosensitivity from normal ambient, artificial, or natural light has been noted but as a precaution patients arc advised not to stay in direct sunlight for periods over half an hour for one week following sensitizer administration. After 48 hours the patient is then exposed to a light bed containing 48 panels, each with 1028 LED's emitting a combination of visible and infra-red light at the frequencies 635 nm and 820 nm. Light bed exposure varies from two sessions of 2 to 15 minutes per day with shorter exposure duration in cases with larger tumor load. Ultrasound is applied using a single maniple at 1 W/cm2 and a frequency of 1 MHz at sites of known malignant disease for 10-30 minutes total. Light and ultrasound activation is repeated on three consecutive days. Ozone auto-haemotherapy (40 IU) is administered immediately before light bed exposure. Further the sensitizer is usually administered after one week for a second treatment cycle. Dexamethasone is administered to some patients with significant tumor load, with dosage titrated on a case-by-case basis.

Other examples are found in Kenyon, Julian N., Richard James Fuller, and Thomas Joseph Lewis. “Activated cancer therapy using light and ultrasound-A case series of sonodynamic photodynamic therapy in 115 patients over a 4 year period.” Current Drug Therapy 4.3 (2009): 179-193 and Wang, Xiaohuai, et al. “Sonodynamic and photodynamic therapy in advanced breast carcinoma: a report of 3 cases.” Integrative Cancer Therapies 8.3 (2009): 283-287.

While this invention has been particularly shown and described with references to preferred embodiments thereof, it will be understood by those skilled in the art that various changes in form and details may be made therein without departing from the scope of the invention encompassed by the appended claims.

1-49. (canceled)

50. A method for treating cancer in a subject using one or more of sonodynamic therapy and photodynamic therapy, the method comprising administering to the subject an agent comprising a mixture a first compound, a second compound, a third compound and a fourth compound, each compound having an independent structural formula IV: 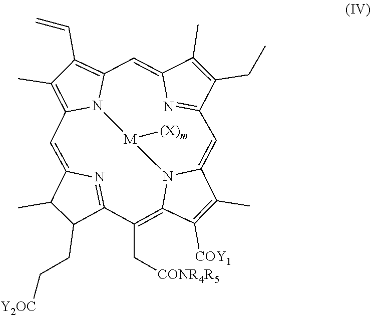
wherein M is Sn(IV), X is Cl, m is 2, Y₁ and Y₂ are OH, and NR₄R₅ is a peptide amide;
wherein the first, second, third and fourth compounds are present in the agent in a weight ratio of approximately 4:2:1:1, and subjecting the subject to one or more of ultrasound and red light.

51. The method according to claim 50, wherein the first compound is Sn(IV) chlorin e6 gly-gly amide dihydroxide sodium salt.

52. The method according to claim 50, wherein the second compound is Sn(IV) chlorin e6 gly-gly-gly-gly amide dihydroxide sodium salt.

53. The method according to claim 50, wherein the third compound is Sn(IV) chlorin e6 Taurine amide dihydroxide sodium salt.

54. The method according to claim 50, wherein the fourth compound is Sn(IV) chlorine e6 L-serine amide dihydroxide sodium salt.

55. The method according to claim 50, wherein the fourth compound is Sn(IV) chlorine e6 lycine amide dihydroxide sodium salt.

56. The method according to claim 50, wherein the first compound is Sn(IV) chlorin e6 gly-gly amide dihydroxide sodium salt, the second compound is Sn(IV) chlorin e6 gly-gly-gly-gly amide dihydroxide sodium salt, the third compound is Sn(IV) chlorin e6 Taurine amide dihydroxide sodium salt, and the fourth compound is Sn(IV) chlorine e6 L-serine amide dihydroxide sodium salt.

57. The method according to claim 50, wherein the first compound is Sn(IV) chlorin e6 gly-gly amide dihydroxide sodium salt, the second compound is Sn(IV) chlorin e6 gly-gly-gly-gly amide dihydroxide sodium salt, the third compound is Sn(IV) chlorin e6 Taurine amide dihydroxide sodium salt, and the fourth compound is Sn(IV) chlorine e6 lycine amide dihydroxide sodium salt.

58. A method for treating cancer in a subject using one or more of sonodynamic therapy and photodynamic therapy, the method comprising administering to the subject an agent comprising a mixture a first compound, a second compound, a third compound a fourth compound, and a fifth compound, each compound having an independent structural formula IV: 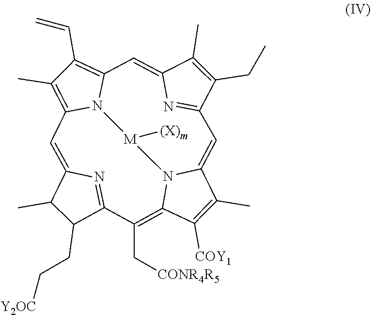
wherein M is Sn(IV), X is Cl, m is 2, Y₁ and Y₂ are OH, and NR₄R₅ is a peptide amide;
wherein the first, second, third and fourth compounds are present in the agent in a weight ratio of approximately 4:2:1:1:1, and subjecting the subject to one or more of ultrasound and red light.

59. The method according to claim 58, wherein the first compound is Sn(IV) chlorin e6 gly-gly amide dihydroxide sodium salt.

60. The method according to claim 58, wherein the second compound is Sn(IV) chlorin e6 gly-gly-gly-gly amide dihydroxide sodium salt.

61. The method according to claim 58, wherein the third compound is Sn(IV) chlorin e6 Taurine amide dihydroxide sodium salt.

62. The method according to claim 58, wherein the fourth compound is Sn(IV) chlorine e6 L-serine amide dihydroxide sodium salt.

63. The method according to claim 58, wherein the fifth compound is Sn(IV) chlorine e6 lycine amide dihydroxide sodium salt.

64. The method according to claim 50, wherein the first compound is Sn(IV) chlorin e6 gly-gly amide dihydroxide sodium salt, the second compound is Sn(IV) chlorin e6 gly-gly-gly-gly amide dihydroxide sodium salt, the third compound is Sn(IV) chlorin e6 Taurine amide dihydroxide sodium salt, the fourth compound is Sn(IV) chlorine e6 lycine amide dihydroxide sodium salt, and the fifth compound is Sn(IV) chlorine e6 lycine amide dihydroxide sodium salt.
